(19) United States
(12) Patent Application Publication
(10) Pub. No.: US 2015/0037833 A1
(43) Pub. Date: Feb. 5, 2015

(54) CONTAINER AND SYSTEM FOR SAMPLE COLLECTION AND PREPARATION

(71) Applicants: Mark James Fisher, Highland Park, IL (US); Sally M. McFall, Evanston, IL (US); Robert D. Hillman, Chicago, IL (US); Zachary J. Walker, Chicago, IL (US); Jacqueline Rene Groves, Chicago, IL (US); Jennifer Reed, Chicago, IL (US); David M. Kelso, Wilmette, IL (US)

(72) Inventors: Mark James Fisher, Highland Park, IL (US); Sally M. McFall, Evanston, IL (US); Robert D. Hillman, Chicago, IL (US); Zachary J. Walker, Chicago, IL (US); Jacqueline Rene Groves, Chicago, IL (US); Jennifer Reed, Chicago, IL (US); David M. Kelso, Wilmette, IL (US)

(73) Assignee: Northwestern University, Evanston, IL (US)

(21) Appl. No.: 14/388,200

(22) PCT Filed: Mar. 27, 2013
(86) PCT No.: PCT/US2013/034168

§ 371 Date: Sep. 25, 2014

Related U.S. Application Data
(60) Provisional application No. 61/616,243, filed on Mar. 27, 2012.

(51) Int. Cl. C12Q 1/24

(52) U.S. Cl. CPC C12Q 1/24
USPC 435/30; 435/283.1

(57) ABSTRACT

The present invention relates to a system for collecting and preparing a body fluid sample, the system comprising a sample container (10) comprising a sample cup (38) for receiving the sample, said sample cup comprising graduated indicator markings (14) corresponding to equal increments of sample volume, a removable lid (16) for sealably covering said sample cup, said lid having an access point which is sealed by a septum, and a removable cap (22) which is effective to cover said access point, and a delivery device for containing a plurality of predetermined reagent doses which are to be added to a sample within the sample container in the predetermined doses relative to the volume of the sample.

### CROSS-REFERENCE TO RELATED APPLICATIONS

This application claims the benefit of priority to U.S. Provisional Application No. 61/616,243, filed Mar. 27, 2012, which is incorporated by reference in its entirety herein.

### STATEMENT REGARDING FEDERALLY SPONSORED RESEARCH OR DEVELOPMENT

This invention was made with government support under grant number U54 EB007949 (Program for Appropriate Technology in Health (PATH) Agreement NIH 1374-02-08459-COL to Northwestern University) awarded by the National Institutes of Health. The government has certain rights in the invention.

### TECHNICAL FIELD

This disclosure is concerned with apparatus and methods for obtaining and preparing body fluid samples for diagnostic testing, such as such as sputum samples to be tested for an analyte such as tuberculosis. In particular, it is concerned with sample preparation apparatus and methods having enhanced safety and simplicity features.

### BACKGROUND

Tuberculosis (TB) is caused by infection of the lungs (in the vast majority of cases) by the bacterium mycobacterium tuberculosis. While both preventable and treatable, TB remains one of the world's leading causes of illness and death. In 2009, an estimated 14 million people were living with active TB, and there were an estimated 1.7 million deaths attributed to TB (WHO, Global Tuberculosis Control 2010). TB affects the developing world disproportionately, with more than 90% of new cases appearing in developing countries.

Reliable clinical diagnosis of the disease in such settings presents a challenge. Chest X-rays, skin tests, and microscopic examination are widely known procedures and generally easy to implement, but they are not sufficiently reliable. The World Health Organization (WHO) reported in 2010 that available blood tests for TB were also giving an unacceptably high number of false negatives and false positives.

Analysis for the bacterium in sputum samples, using nucleic acid amplification technology, is the currently preferred standard for accurate diagnosis of TB. However, the collected sputum sample requires dilution and other pretreatment, which raises the risk of exposing technicians to contaminated samples. In the developing world, few clinics or hospitals have well-functioning safety hoods for handling TB samples, due to lack of availability, lack of floor space, and/or expense.

Thus, there is a need for sample collection systems that both reduce exposure of health care workers and also simplify the addition of thinning agents and other reagents for sample preparation.

### SUMMARY

The following aspects and embodiments described and illustrated below are meant to be exemplary and illustrative, and are no way intended to be limiting in scope.

Disclosed herein, in one aspect, is a system for collecting and preparing a body fluid sample, the system comprising:

(a) a sample container comprising:
(i) a sample cup for receiving the sample, said sample cup comprising graduated indicator markings corresponding to equal increments of sample volume,
(ii) a removable lid for sealably covering said sample cup, said lid having an access point which is sealed by a septum, and
(iii) a removable cap which is effective to cover said access point, and

(b) a delivery device for containing a plurality of predetermined reagent doses which are to be added to a sample within the sample container in the predetermined doses relative to the volume of the sample,

wherein the reagent doses are for insertion into the sample container through the septum, and

wherein the predetermined reagent doses correspond to the corresponding indicator marking on said delivery device such that the number of predetermined doses of reagent a to be added to a volume of sample corresponds to the indicator marking on the sample cup.

In one embodiment, the septum comprises one or more slits allowing insertion of the predetermined dose through the septum. The predetermined dose may be inserted directly through the septum as in the case of a solid dosage form or by penetration of a delivery device.

In one embodiment, the plurality of predetermined reagent doses are in the form of a solution. In a further embodiment, the delivery device is configured or able to penetrate the septum. In another embodiment, the delivery device includes graduated indicator markings that indicate a volume of the predetermined doses to be added to a volume of sample corresponding to the markings on the sample cup. In other embodiments, the indicator markings on the sample cup and the delivery device are consecutively numbered graduated markings. In one preferred embodiment, the consecutively numbered graduated markings on the sample cup and on the delivery device employ unitless numbers. In different embodiments, the delivery device may be a pipette, a sealed package having a tip which can be removed or pierced and inserted through the septum, or a syringe.

Typically, the access point is an opening in the lid from which the septum is removable. Alternatively, the septum may be attached to or be a part of the lid (in which case the septum and the access point may be considered synonymous).

In a preferred embodiment, the interior surface of the sample cup is at least partially conical or frustoconical in shape. The sample container may further comprise members extending from the exterior sides of the sample cup to support the container in an upright position. In another preferred embodiment, the sample cup is sufficiently translucent to allow a volume of sample contained within the sample cup to be visible to an observer.

The components of the system may be provided in kit form. In such cases, the system preferably further includes the reagent solution and/or solid reagent doses as the predetermined reagent dose(s). In one embodiment, the reagent solution is contained within the sealed package that serves as the delivery device. The reagent may comprise, in various embodiments, one or more cell lysing reagents and/or one or more mucolytic reagents for treatment of the sample, particularly a sputum sample. In an embodiment, the cell lysing reagent is a detergent. In another embodiment, the mucolytic reagent is a proteinase such as proteinase K.

Also provided by the disclosure herein is a sample container as described above, for collecting and preparing a body fluid sample, the container comprising:

(i) a sample cup for receiving the sample, the sample cup comprising consecutively numbered graduated markings corresponding to equal increments of sample volume,

(ii) a removable lid for sealably covering the sample cup, the lid having an access point which is sealed by a septum, and

(iii) a removable cap which is effective to cover the access point,

wherein the consecutively numbered graduated markings on the sample cup employ unitless numbers.

Preferably, the sample cup is sufficiently translucent to allow a volume of sample contained within the cup to be visible to an observer. In one embodiment, the interior surface of the sample cup is conical or frustoconical in shape. In different embodiments, the access point is an opening in the lid from which the septum is removable, or the septum may be attached to or be a part of the lid.

Also disclosed herein is a related method for preparing a body fluid sample, the method comprising:

adding a reagent to a body fluid sample within a sample container, in a predetermined amount relative to the volume of said sample,

wherein said sample container comprises (i) a sample cup for receiving the sample, comprising graduated indicator markings corresponding to equal increments of sample volume, (ii) a removable lid, containing an access point which is sealed by a septum, and (iii) a removable cap effective to cover said access point;

wherein said reagent is added as a predetermined amount through said septum, and

wherein the predetermined amount added to the sample container corresponds to the volume of sample collected in the sample container.

In particular, the adding of solution comprises the steps of:

observing the level of sample within the sample cup;

assigning a number or other indicator to the sample volume, corresponding to the level of sample in the cup with respect to the graduated indicator markings on the sample cup, and

adding to the sample from the delivery device, a volume of reagent solution or amount/number of solid reagent which corresponds to the same number with respect to said graduated indicator markings on the delivery device.

In an embodiment, the reagent is a solution contained within a delivery device including markings for the amount of reagent to add to the sample container corresponding to the graduated markings on the sample container. In another embodiment, the reagent is a predetermined amount of the reagent as a discrete solid, where the number of discrete solids added to the sample container correspond to the graduated indicator markings on the sample cup.

In an embodiment, the indicator markings on the sample cup and/or on the delivery device are consecutively numbered graduated markings. In one preferred embodiment, the consecutively numbered graduated markings on the sample cup and/or on the delivery device employ unitless numbers. In one embodiment, the volume of reagent solution or amount of solid reagent dosage added is not in a 1:1 ratio to the sample volume. Preferably, the interior surface of the sample cup is conical or frustoconical in shape, and the sample cup is sufficiently translucent to allow a volume of sample contained within the sample cup to be visible to an observer.

As disclosed above, the device septum preferably comprises one or more slits allowing penetration of the septum by the delivery device, which may be, for example, a pipette, a sealed package having a tip which can be removed or pierced and inserted through the septum, or a syringe. In another embodiment, the one or more slits allow penetration of the septum by one or more solid reagent doses.

In a preferred embodiment of the method, the body fluid sample is a sputum sample. The reagent solution added to the sample may contain, in various embodiments, cell lysing reagents and/or mucolytic reagents. In embodiments, the cell lysing reagent is a detergent. In other embodiments, the mucolytic reagent is a proteinase such as proteinase K.

In further embodiments of the method, the method further comprises isolating nucleic acids from the sample, and may further comprise amplifying one or more target nucleic acids from the isolated nucleic acids. Such amplification may use any amplification method known in the art; examples include, but are not limited to, PCR, RT (real time)-PCR, RT (reverse transcriptase)-PCR, and isothermal techniques such as nucleic acid sequence based amplification (NASBA), transcription mediated amplification (TMA), strand displacement amplification (SDA), ligase chain reaction (LCR), and helicase dependent amplification (SDA).

In a preferred embodiment, the one or more target nucleic acids is characteristic of Mycobacterium tuberculosis, and the method is used to determine the presence or absence of Mycobacterium tuberculosis in a body fluid sample, particularly a sputum sample.

Also disclosed herein is a further method for preparing a body fluid sample: the method comprising:

adding a reagent solution to a body fluid sample within a sample container such as disclosed herein, in a predetermined volume relative to the volume of the sample,

wherein the sample container comprises, as disclosed above, (i) a sample cup for receiving the sample, comprising consecutively numbered graduated markings corresponding to equal increments of sample volume, (ii) a removable lid, containing an access point which is sealed by a septum, and (iii) a removable cap effective to cover the access point;

wherein the reagent solution is added using a delivery device which is able to penetrate the septum, and which comprises consecutively numbered graduated markings corresponding to equal increments of reagent solution volume,

and wherein the predetermined volume added to a volume of sample which corresponds to a given number on the sample cup is the volume of reagent solution which corresponds to the same given number on the delivery device. Additional aspects and advantages of the present devices and methods are set forth in the following description and claims, particularly when considered in conjunction with the accompanying examples and drawings.

### BRIEF DESCRIPTION OF THE DRAWINGS

FIG. 1 illustrates one embodiment of a sample container as disclosed herein;

FIG. 2 shows a sample container as illustrated in FIG. 1 in cross-section; and

FIGS. 3A-3B show embodiments of delivery devices as disclosed herein, where FIG. 3A shows a pipette and FIG. 3B shows a sealed pouch.

FIG. 4 is a graph showing relative extraction for a standard buffer or a 10% dilution.

FIG. 5 is an image of a time course of sputum processed with proteinase K digestion buffer at 0 minutes, 5 minutes, 10 minutes, and 15 minutes.

### DETAILED DESCRIPTION

### I. Definitions

Before the present methods and compositions are described, it is to be understood that this disclosure is not limited to particular embodiments described, as such may, of course, vary. Several embodiments of the present disclosure are described in detail hereinafter. These embodiments may take many different forms and should not be construed as limited to those embodiments explicitly set forth herein. Rather, these embodiments are provided so that this disclosure will be thorough and complete, and will fully convey the scope of the present disclosure to those skilled in the art. It is also to be understood that the terminology used herein is for the purpose of describing particular embodiments only, and is not intended to be limiting, since the scope of the invention will be limited only by the appended claims.

All patents, applications, published applications and other publications referred to herein are incorporated by reference in their entirety.

Terms and abbreviations not defined should be accorded their ordinary meaning as used in the art. As used herein, the following terms are intended to have the following meanings:

As used herein, the singular forms “a,” “an,” and “the” encompass plural referents unless the context clearly dictates otherwise. Thus, for example, reference to “a protein” includes a plurality of such proteins and reference to “the formulation” includes reference to one or more formulations and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed by this disclosure. The upper and lower limits of these smaller ranges may independently be included or excluded in the range, and each range where either, neither or both limits are included in the smaller ranges is also encompassed by this disclosure, subject to any specifically excluded limit in the stated range. Where the stated range includes one or both of the limits, ranges excluding either or both of those included limits are also within the scope of this disclosure. For example, if a range of 5 to 10 minutes is stated, it is intended that 6 min., 7 min., 8 min., and 9 min. are also explicitly disclosed, as well as the range of values greater than or equal to 5 min. and the range of values less than or equal to 10 min.

“Detection” of a target nucleic acid or analyte refers to determining the presence or the absence of the nucleic acid or analyte in a sample, where absence refers to a zero level or an undetectable level.

As pertains to the present disclosure, a biological fluid or “body fluid” can be, unless otherwise indicated, a solid, or semi-solid sample, including feces, biopsy specimens, skin, nails, and hair, or a liquid sample, such as urine, saliva, sputum, mucous, blood, blood components such as plasma or serum, amniotic fluid, semen, vaginal secretions, tears, spinal fluid, washings, and other bodily fluids. Included among the sample are swab specimens from, e.g., the cervix, urethra, nostril, and throat. In particular embodiments, the sample is a sputum sample.

### II. Sample Collection and Preparation System

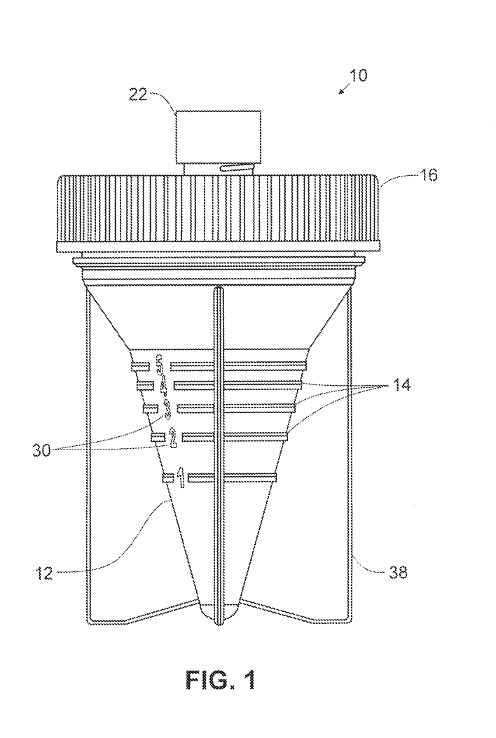

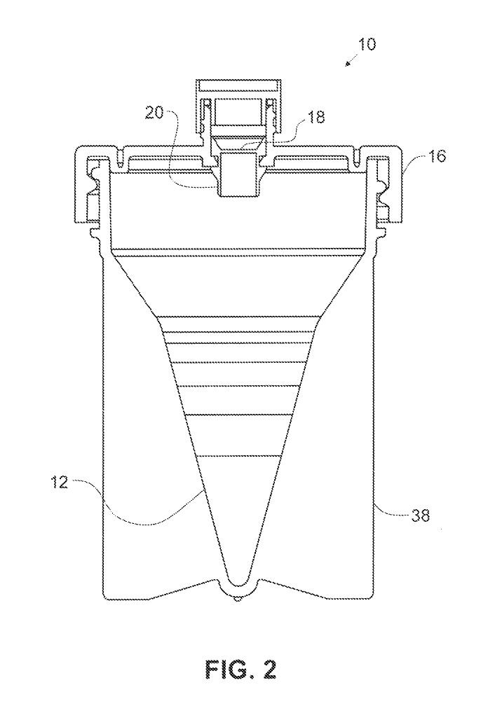

Provided herein is a sample collection container for collecting a body fluid sample and for preparing the sample for diagnostic testing, e.g. by a nucleic acid assay. One embodiment of such a sample container is illustrated in FIGS. 1-2. As shown in FIG. 2, the sample container 10 comprises a sample cup 12 for receiving the sample, a removable lid 16 for sealably covering the sample cup, and a removable cap 22. The lid 16 and cap 22 are typically screw caps attached by threads, as partially shown in the drawings. It will be appreciated that the lid and cap may be snap-on type and include a ridge or projection(s) to secure the lid or cap. The lid 16 has an access point, typically at least one central opening which is sealed by a septum 18 (not visible in FIG. 1). The septum is preferably slit to allow penetration by e.g. a solution delivery or extraction device which is not a sharps device.

The septum is typically molded as a separate part which is inserted into the lid. It will be appreciated that the septum may be removable from the lid. In embodiments, the septum may be attached to the lid, removably or otherwise. In other embodiments, the septum is integral with the lid. In the embodiment shown in cross-sectional FIG. 2, the septum material (typically rubber or a polymer) also forms a cylindrical extension 20 extending a short distance into the interior of the container. However, such an extension is optional.

Alternatively, the lid and septum may be molded together, in which case the access point comprises, for example, an opening with an overmolded rubber septum, or a very thin section of the same plastic material making up the lid, either of which is preferably slit to allow access. However, a removable septum is generally preferred, since this allows the device to be provided to the user with the septum removed, thus permitting access to the sample, e.g. with a smear stick or probe, for obtaining a microscope smear sample, without removing the entire lid of the sample container. (While amplification assays are preferred from the standpoint of accuracy, microscopic smear examination is still widely used in developing countries for first-pass TB diagnosis.)

The sample cup 12 comprises, along at least a portion of its exterior surface, graduated indicator markings 14 which correspond to equal increments of sample volume. As shown in FIG. 2, the markings may be indicator lines without a numerical marking. In embodiments, the indicator markings are consecutively numbered graduated markings 14. Adjacent to the graduated markings are consecutive numerals 30; these are preferably unitless numbers, as described further below. Preferably, the sample cup is sufficiently translucent to allow a volume of sample contained within the cup to be visible to an observer.

In a preferred embodiment, the interior surface of the sample cup 12 is at least partially conical or frustoconical in shape, as shown in the Figures, to allow more accurate measurement of smaller samples. The sample container may also comprise members 38 extending from the exterior sides of the sample cup, to support the container in an upright position.

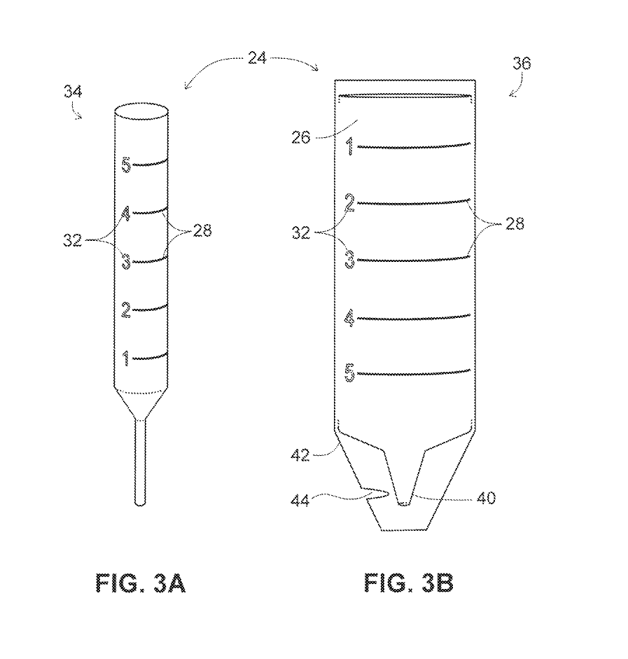

Also provided herein, for use with the sample container, is a delivery device 24 for delivering a diluent and/or reagent solution to the sample within the container, preferably in a predetermined volume relative to the volume of sample (e.g., 2 ml of diluent/reagent to 1 ml of sample). The delivery device is preferably a non-sharps device formed of a stable plastic material, such as a plastic pipette 34 (FIG. 3A) or a sealed pouch 36 having a dispensing tip 40 (FIG. 3B). In the embodiment shown, the pouch includes a flange 42 with a notch 44 to guide the opening of the pouch.

Other types of sealed packages may be used in lieu of the pouch of FIG. 3B, e.g., a thermoformed blister package or a blow-fill-seal container, such as is commonly used for packaging of sterile pharmaceuticals. In any case, the sealed package has a dispensing end that can be pierced, broken, torn or cut off to allow dispensing of the contents, and it is preferably constructed such that liquid does not dispense until significant pressure (i.e. more than is necessary to open the dispensing end of the package and insert it through the septum 18) is applied to the package. In one embodiment, the sealed package has sufficient rigidity to prevent premature dispensing.

In a manner similar to the sample cup 12, the dispensing device comprises, along at least a portion of its exterior surface, graduated markings 28 which correspond to equal increments of solution volume. In embodiments, the markings are consecutively numbered. Adjacent to the graduated markings are consecutive numerals 32; as for the sample cup, these are preferably unitless numbers.

An advantage of the sealed pouch (or other sealed package) is that prepared diluent/reagent solution 26 can be supplied prepackaged in the pouch or other sealed package, thus reducing the need for technicians to manipulate solutions. Thus, in a kit comprising the sample container and sample dispensing device, the diluent/reagent solution can be provided within the sealed package. Alternatively, the diluent/reagent solution may be provided in a separate container if the dispensing device 24 is a pipette.

The concentration of the diluent/reagent solution is such that a quantity corresponding to a given numeral 32 or other indicator on the delivery device is the appropriate quantity for use with a volume of raw sample corresponding to the same numeral 30 on the sample cup. (Intermediate numbers can be estimated and the same correspondence made.)

In one embodiment, the volumes of sample and solution used are not in a 1:1 correspondence, even though the numbers on the different components (the sample cup and delivery device) match. For example, the ratio of actual volumes used may be 2:1, 0.5:1, or various other ratios. Of course, a 1:1 ratio may also be used.

In another embodiment, the delivery device is a container or holder for a plurality of solid reagent doses as described further below. The delivery device in this embodiment may include a dispensing tip or end sufficiently sized to allow the solid reagent dose to pass through the septum and into the sample cup. In other embodiments, the solid reagent dose is removed from the deliver device and inserted through the septum and into the sample cup.

Instructions for carrying out the described sample-to-reagent correspondence would typically be provided with a kit. Such a kit would typically comprise the sample container, the delivery device, and, preferably, the diluent/reagent solution/solid reagent.

The provided diluent/reagent solution may vary, depending on the desired treatment of the sample fluid collected in the sample cup. For example, sputum samples, which are thick and difficult to handle, are conventionally treated with sodium hydroxide solution for initial dilution and liquefaction; this treatment also kills non-TB bacteria. The sample collection and preparation system may be used to prepare samples for culturing or for nucleic acid amplification and analysis.

When the sample is to be prepared for nucleic acid amplification, cell lysing and/or mucolytic or proteolytic reagents may be provided. A kit for nucleic acid analysis may also include, in separate containers, amplification primers and other amplification reagents, to be used in accordance with known procedures. Such amplification may use any amplification method known in the art; examples include, but are not limited to, PCR, RT (real time)-PCR, RT (reverse transcriptase)-PCR, and isothermal techniques such as nucleic acid sequence based amplification (NASBA), transcription mediated amplification (TMA), strand displacement amplification (SDA), ligase chain reaction (LCR), and helicase dependent amplification (SDA).

In an embodiment, the reagent is a protease digestion buffer comprising a cell lysing reagent and a protease. In an embodiment, the cell lysing reagent is a detergent. Any suitable detergent is acceptable such as, for example, an anionic detergent such as sodium dodecyl sulfate (SDS) or cationic detergents. In an embodiment, the buffer includes a reagent for digestion of proteins such as a protease. One suitable protease is Proteinase K. In preferred embodiments, the buffer includes agents to stabilize the protease. In one exemplary embodiment, the buffer includes an activator such as CaCl₂ to activate the protease through increased stability and a reagent to maintain the buffer pH in an effective range. In an embodiment, where the protease is Proteinase K, the buffer reagent is Tris-HCl to maintain the buffer pH at about 8.0 for maximum proteinase K activity. Where the activator is CaCl₂, the buffer preferably includes a reagent for inhibition of calcium-dependent nucleases that could digest the target DNA. In an exemplary embodiment, the inhibitor is EDTA. One advantage of the protease digestion buffer is that the sputum sample is sterilized, thereby reducing infection risk for clinical workers. While the sample may not be used for bacterial growth analysis, it is easily analyzed for the presence of nucleic acids. Another advantage of the protease digestion buffer is that it reduces or prevents false negatives caused by clumping of the bacteria in the specimen. Lysing and mixing of the specimens provides an equal or nearly equal concentration of nucleic acid throughout the sample.

In another embodiment, the reagent is a solid reagent comprising a cell lysing reagent and a protease. One advantage of the dried, solid reagent is extended stability, especially at higher temperatures. The dried reagent is preferably shelf stable for extended periods of time. In one embodiment, the dried reagent is shelf stable for a longer period of time than a corresponding reagent solution. In embodiments, the reagent is stable for about 1-12 months. In non-limiting embodiments, the reagent is stable for about 1-2 months, about 1-4 months, about 1-6 months, about 2-4 months, about 2-6 months, about 2-12 months, about 4-6 months, about 4-12 months, about 6-12 months or longer. In particular, but not limiting embodiments, the dried reagent is stable for at least about 1 month, about 2 months, about 4 months, about 6 months, about 8 months, about 10 months, about 12 months, or longer. In another embodiment, the dried reagent is shelf stable at higher temperatures. This is particularly advantageous for use of the reagent in areas without extensive refrigeration. In embodiments, the dried reagent is shelf stable at a temperature of at least about 25-60° C. In other embodiments, the dried reagent is shelf stable at a temperature of at least about 25-55° C., or at least about 40-55° C. In particular, but not limiting embodiments, the dried reagent is shelf stable at about 40-55° C. for at least about 1-12 months or 1-6 months including the time periods described above. Another advantage is increased safety in handling the reagents. Proteases can be dangerous with prolonged skin contact. A solid, dry dosage form prevents a liquid spill that may contact an extended skin area as well as provides for limited skin exposure to the reagents.

In a preferred embodiment, the dried reagent is prepared by freeze-drying the components alone or together. Where the components are freeze-dried separately, the resulting components may be mixed and formed into a solid dosage form. In preferred embodiments, the dried reagent includes the same or similar ingredients as the protease digestion buffer described above. In one embodiment, protease K with 25 mM HEPES (pH 8.0), 5 mM CaCl₂, and 20 mg/ml trehalose are freeze dried. SDS is freeze-dried separately and the components mixed. An advantage of a solid reagent dosage is that all reagents are contained within a single dosage form. A clinician does not need to measure the reagents individually thereby reducing the potential for error. Further, the dosage form has a single storage requirement, as opposed to multiple storage requirements for the individual reagents. The dried reagent may be individually packaged or packaged together in a delivery device. The dried reagent may be formed in any suitable form including, but not limited to, a tablet, capsule, pill, etc. The dried reagent may further comprise a protective coating such as a gel coating.

In a preferred embodiment, the one or more target nucleic acids is characteristic of mycobacterium tuberculosis, and the method is used to determine the presence or absence of mycobacterium tuberculosis in a body fluid sample, particularly a sputum sample.

### III. Method of Sample Collection and Preparation

Also disclosed herein is a method of preparing a body fluid sample using a sample container and dispensing device as described above. The sample cup 12 is filled by the patient by removal of the cover 16, which is generally a plastic screw cap. If necessary, repeated deposits are made. The interior surface of the sample cup 12 is preferably conical or frustoconical in shape, so that accurate volume measurement is possible at both small volumes and larger volumes. Typically, the sample cup is designed to hold 1-5 ml or 1-10 ml of accumulated sample. As noted above, however, the indicia 30 on the cup generally do not include volume units.

At the clinic, the level of the sample, typically sputum, in the sample cup 12 is noted. In particular, its correspondence to the marker indicia 30 is noted, and an intermediate number is estimated if necessary. (In this sense, when referring to a “volume of sample which corresponds to a given number on the sample cup” herein, the “given number” need not be a whole number, and can be an intermediate or fractional number.) The small cap 22 is removed by a clinical worker, exposing (in one embodiment) the septum 18 which seals the opening in cover 16. Preferably, the septum as provided is slit to allow access via a non-sharp instrument such as a plastic pipette or the dried reagent; in another embodiment, the septum is solid and is pierced using a syringe. The septum prevents aerosols from escaping the sample cup when the cap is removed and when the contents are accessed.

For sample preparation, the diluent/reagent solution of appropriate concentration, as described above, is preferably provided with the sample container and delivery device, either in a container to be drawn up into the pipette 34 or prepacked in a sealed contained such as pouch 36. In a less preferred embodiment, a diluent/reagent solution having the appropriate concentration is prepared at the clinic and then utilized, for example, drawn up into pipette 34. In another embodiment, the appropriate number of discrete dried reagents are added to the sample cup based on the amount of sample collected in the cup.

As defined herein, the “appropriate concentration” of the diluent/reagent solution is such that a quantity corresponding to a given numeral (32) on a delivery device as described herein is the correct predetermined quantity for use with a volume of raw sample corresponding to the same numeral (30) on a sample cup as described herein.

With reference to the number previously associated with the level of sample in sample cup 12, a volume of diluent/reagent solution corresponding to the same number (32) on delivery device 24 is then added to the sample cup, via septum 18. For example, in one embodiment, the desired volume ratio of diluent/reagent solution to sample is 2:1. In this embodiment, each marking 14 on sample cup 12 could correspond to a 1 ml increment, in which case the markings 28 on the delivery device (e.g. pipette or pouch) would correspond to 2 ml increments. If the sample volume level corresponds to the number “3”, for example, then an amount of the pouch or pipette contents corresponding to the number “3” is used. Thus, for example, diluent/reagent solution is dispensed from the pouch until the liquid level in the packet reaches the appropriate level number; alternatively, an amount of solution corresponding to the appropriate level number is drawn up into the pipette and then dispensed.

The system as disclosed has a number of advantages. Not only does the system protect the technician from exposure to the sample, but it also allows an accurate predetermined amount of diluent/reagent solution to be added, for any predetermined ratio of components, without the need for calculations on the part of the technician.

### Example 1

### Sputum Collection

Sample collection containers were provided to 98 human patients suspected of Mycobacterium tuberculosis infection to determine the ease of use and effectiveness in obtaining a sample with sufficient volume (≧1 ml) for testing. 93 of the subjects produced at least some sample in the container with the amounts being shown in Table 1. All of these containers had the lid attached correctly and none of the containers showed any leakage. Thus, the containers were easy and effective for the patients to use. Further, the patients found the containers easy to hold and easy to close.

The containers were effective for obtaining a sufficient sample size. Of the patients that produced at least some sample, 93.7% of patients produced a volume of sputum ≧1 ml in the containers (87/93).

TABLE 1
Sputum Collection

| | | |
|---|---|---|
| Volume | #Containers | % |

| | | | |
|---|---|---|---|
| <1 | mL | 1 | 1 |
| 1 | mL | 54 | 58 |
| 2 | mL | 23 | 24 |
| 3 | mL | 8 | 8 |
| 5 | mL | 2 | 2 |

### Example 2

### Sputum Digestion and Sterilization

A 2× sputum protease digestion buffer comprising 60 mM Tris, pH 8.0, 2 mM CaCl₂, 2% SDS, and 1 mg/mL proteinase K was prepared.

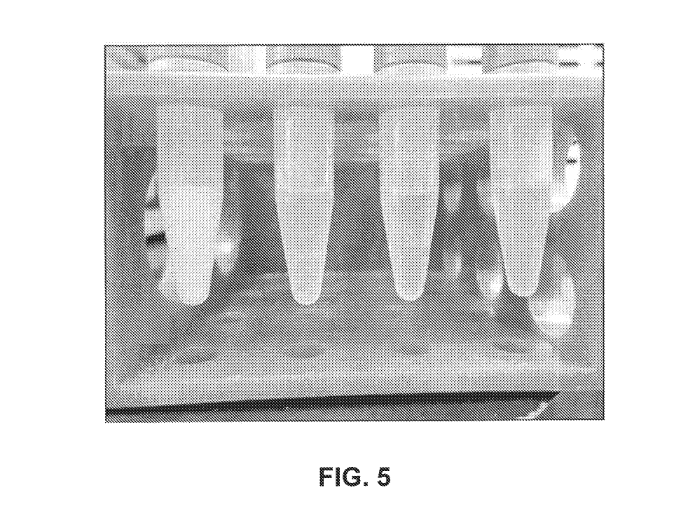

1 mL of raw sputum was added to 1 mL of the 2× sputum protease digestion buffer in a 15 mL Falcon tube. The sputum and buffer was heated to 55° C. in a Benchmark Multitherm Shaker for about 7.5 minutes with shaking at 1000 rpm. The solution was then heated to 95° C. for 10-20 minutes with shaking at 1000 rpm. The resulting solution was homogenous and easily pipetted. FIG. 5 is a time course of sputum processed with the proteinase K digestion buffer at (from left to right) 0 minutes, 5 minutes, 10 minutes, and 15 minutes. Sputum appearance changes from opaque, viscous liquid to a free-flowing translucent liquid. There were no difficulties in pipetting or heterogeneity of specimens were observed with even the thickest specimens.

Heat killing the organisms in the sample before a clinician removes samples for analysis prevents the operator from exposure to live organisms such as Mycobacterium tuberculosis. Further, the reagent buffer thins and homogenizes the specimen making it easier to pipette and/or measure accurately.

### Example 3

### Sputum Sterilization

1 mL of raw sputum was spiked with 1E7 viable organisms and 1 mL of a 2× protease digestion buffer as described in Example 2 was added. The sputum and buffer was heated to 55° C. for about 7.5 minutes with shaking at 1000 rpm. The temperature was raised to 95° C. for 0, 3, 5, 10, 20 or 30 minutes with shaking at 1000 rpm. The samples were centrifuged at 3000 rpm for 15 minutes and the supernatant discarded. The pellet was washed with phosphate buffered saline (PBS) and centrifuged at 3000 rpm for 15 minutes. The supernatant was discarded and the pellet resuspended in 100 μl PBS. Serial dilutions 10E-1 to 10E-4 were prepared and the dilutions were plated in triplicate. The dilutions were incubated at 37° C. and inspected weekly for growth with the results shown in Table 2.

TABLE 2
Bacterial Growth

| Raised Temperature | |
|---|---|
| Time (min) | Growth |
| 0 | growth |
| 3 | no growth |
| 5 | growth |
| 10 | no growth |
| 20 | no growth |
| 30 | no growth |

These results show that raising the temperature to 95° C. for at least about 10 minutes is sufficient to kill the added bacteria (MTB).

### Example 4

### Effect of Buffer Dilution

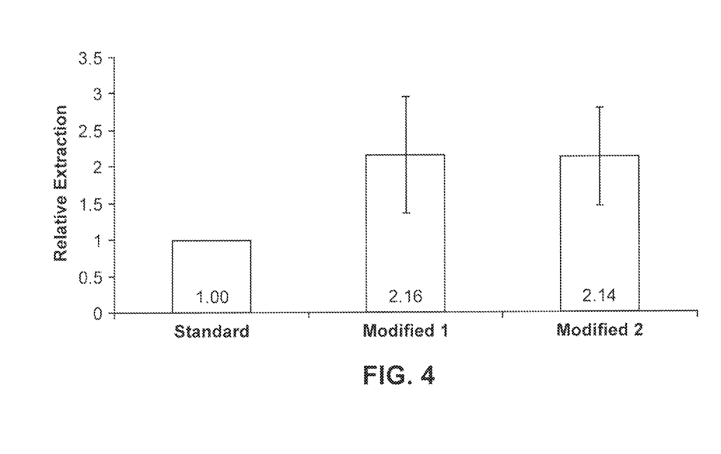

To test the effect of dilution of the buffer, a standard digestion buffer was prepared and a 2× digestion buffer was prepared in accord with Example 1. The standard buffer was added to a sputum sample at a 100% dilution (1 mL sputum to 1 mL buffer). A 10% dilution was prepared using the concentrated buffer (0.9 mL sputum to 0.1 mL buffer). The reagent constituents (proteinase K, CaCl₂ and SDS) were kept at the same concentration for each dilution. The relative extraction was measured with the results shown in FIG. 4. The 100% dilution with the standard buffer was set as 1 and the two modified samples are expressed as a ratio of the standard method. As seen from FIG. 4, using the 10% dilution produced two times better results than the standard buffer.

### Example 5

### Dry Digestion Reagent

A dry reagent for digestion and sterilization of sputum is formed by freeze drying proteinase K with 25 mM HEPES, pH 8.0, 5 mM CaCl₂, and 20 mg/ml trehalose. 2% SDS is freeze dried and mixed with the proteinase K composition. The resulting digestion reagent is formed into a pill, tablet, or capsule. The resulting pills, tablet, or capsules may be stored in strips sealed with aluminum foil or may be stored in another suitable container.

These and other applications and implementations will be apparent in view of the disclosure. Such modifications, substitutions and alternatives can be made without departing from the spirit and scope of the invention, which should be determined from the appended claims. While the present device, system, and method have been described with reference to several embodiments and uses, and several drawings, it will be appreciated that features and variations illustrated or described with respect to different embodiments, uses, and drawings can be combined in a single embodiment.

1. A system for collecting and preparing a body fluid sample, the system comprising:
(a) a sample container comprising:
(i) a sample cup for receiving the sample, said sample cup comprising graduated indicator markings corresponding to equal increments of sample volume,
(ii) a removable lid for sealably covering said sample cup, said lid having an access point which is sealed by a septum, and
(iii) a removable cap which is effective to cover said access point, and
(b) a delivery device for containing a plurality of predetermined reagent doses which are to be added to a sample within the sample container in the predetermined doses relative to the volume of the sample,
wherein the reagent doses are for insertion into the sample container through the septum, and
wherein the predetermined reagent doses correspond to the corresponding indicator marking on said delivery device such that the number of predetermined doses of reagent a to be added to a volume of sample corresponds to the indicator marking on the sample cup.

2. The system of claim 1, wherein the septum comprises one or more slits allowing insertion of the predetermined dose through the septum.

3. The system of claim 1, wherein the plurality of predetermined reagent doses are in the form of a solution, the delivery device being able to penetrate the septum, and comprising graduated indicator markings that indicate a volume of the predetermined doses to be added to a volume of sample corresponding to the markings on the sample cup.

4. The system of claim 3, wherein the indicator markings on the sample cup and the delivery device are consecutively numbered graduated markings.

5. The system of claim 4, wherein the consecutively numbered graduated markings on the sample cup and on the delivery device employ unitless numbers.

6. The system of claim 3, wherein the one or more slits allow penetration of the septum by the delivery device.

7. The system of claim 6, wherein the delivery device is a pipette.

8. The system of claim 7, wherein the delivery device is a sealed package having a tip which can be removed or pierced and inserted through said septum.

9. The system of claim 1, wherein the delivery device is a syringe.

10. The system of claim 2, wherein the predetermined reagent dose is a solid dosage form and the one or more slits allow penetration of the solid dosage form.

11. The system of claim 1, wherein said access point is an opening in said lid from which said septum is removable.

12. The system of claim 1, wherein said septum is attached to or is part of said lid.

13. The system of claim 1, wherein the interior surface of the sample cup is conical or frustoconical in shape.

14. (canceled)

15. The system of claim 1, further comprising the plurality of predetermined reagent doses.

16. The system of claim 15, wherein said plurality of predetermined reagent doses comprises at least one cell lysing reagent.

17. The system of claim 16, wherein the at least one cell lysing reagents is a detergent.

18. The system of claim 15, wherein said plurality of predetermined reagent doses comprises at least one mucolytic reagent.

19. The system of claim 18, wherein said mucolytic reagent is a proteinase.

20. A method for preparing a body fluid sample, the method comprising:
adding a reagent to a body fluid sample within a sample container, in a predetermined amount relative to the volume of said sample,
wherein said sample container comprises (i) a sample cup for receiving the sample, comprising graduated indicator markings corresponding to equal increments of sample volume, (ii) a removable lid, containing an access point which is sealed by a septum, and (iii) a removable cap effective to cover said access point;
wherein said reagent is added as a predetermined amount through said septum, and
wherein the predetermined amount added to the sample container corresponds to the volume of sample collected in the sample container.

21. The method of claim 20, wherein the reagent is a solution contained within a delivery device including markings for the amount of reagent to add to the sample container corresponding to the graduated markings on the sample container.

22. The method of claim 20, the reagent is a predetermined amount of the reagent as a discrete solid, where the number of discrete solids added to the sample container correspond to the graduated indicator markings on the sample cup.

23. The method of claim 21, wherein said adding comprises:
observing the level of sample within the sample cup;
assigning a number to the sample volume, corresponding to the level of sample in the cup with respect to said graduated indicator markings on the sample cup, and
adding to the sample from the delivery device, a volume of reagent solution or amount of a solid reagent dosage which corresponds to the same number with respect to said graduated indicator markings on the delivery device.

24. The method of claim 23, wherein the graduated indicator marking are consecutively numbered markings.

25. The method of claim 24, wherein the consecutively numbered graduated markings on the sample cup and on the delivery device employ unitless numbers.

26. The method of claim 21, wherein the volume of reagent solution added is not in a 1:1 ratio to said sample volume.

27-39. (canceled)

40. A sample container for collecting and preparing a body fluid sample, the container comprising:
(i) a sample cup for receiving the sample, said sample cup comprising consecutively numbered graduated markings corresponding to equal increments of sample volume,
(ii) a removable lid for sealably covering said sample cup, said lid having an access point which is sealed by a septum, and
(iii) a removable cap which is effective to cover said access point,
wherein the consecutively numbered graduated markings on the sample cup employ unitless numbers.

41-44. (canceled)
